(19) United States
(12) Patent Application Publication
(10) Pub. No.: US 2023/0389787 A1
(43) Pub. Date: Dec. 7, 2023

(54) ENDOSCOPE CLEANER OPERABLE TO BE COUPLED TO A TROCAR

(71) Applicants: Clark B. FOSTER, Mission Viejo, CA (US); Gerald Jay SANDERS, Mission Viejo, CA (US); Gerald FEUER, Atlanta, GA (US)

(72) Inventors: Clark B. FOSTER, Mission Viejo, CA (US); Gerald Jay SANDERS, Mission Viejo, CA (US); Gerald FEUER, Atlanta, GA (US)

(21) Appl. No.: 18/034,161

(22) PCT Filed: Oct. 28, 2021
(86) PCT No.: PCT/US2021/057137

§ 371 Date: Apr. 27, 2023

Related U.S. Application Data
(60) Provisional application No. 63/106,748, filed on Oct. 28, 2020.

(51) Int. Cl. A61B 1/12; A61B 1/05; A61B 17/34

(52) U.S. Cl. CPC A61B 1/122; A61B 1/05; A61B 17/3417; A61B 2017/3454

(57) ABSTRACT

Endoscope or camera cleaning device having a jaw extending away from a tube. Sheath extends away from the tube. Sheath has sidewalls corresponding to another portion of the circumference of the tube and the sidewalls of the sheath and the sidewalls of the jaw being biased towards each other through at least one elastic member. Slider is coupled to the jaw and operable to translate with respect to the jaw. Wiper blade is coupled to the slider. Wiper blade has a lower edge that extends away from the jaw and extends beyond a centerline with respect to the tube. Wiper blade is operable to be engaged by a camera traversing through the attachment tube. Wiper blade crosses the centerline and allows the camera to pass beyond the wiper, which is operable to remove fluids and/or substances from the endoscope or camera.

### CROSS-REFERENCE TO RELATED APPLICATION

The present application claims the benefit of and priority to U.S. Provisional Patent Application No. 63/106,748 filed Oct. 28, 2020, the contents of which are incorporated by reference herein.

### FIELD

The present disclosure relates generally to a lens cleaner for an endoscope. In at least one example, the present disclosure relates to an assembly to be coupled to a trocar and operable to clean an endoscope.

### BRIEF DESCRIPTION OF THE DRAWINGS

Implementations of the present technology will now be described, by way of example only, with reference to the attached figures, wherein:

FIG. 1 illustrates an example device with a camera extended past the wiper and the elastic member in the extended state.

FIG. 2 illustrates a camera extending through a trocar without the device attached thereto.

FIG. 3 illustrates a camera that has been contaminated and being retracted for cleaning.

FIG. 4 illustrates the contamination that is left in a twin blade design of the prior art.

FIGS. 5A-5C illustrate examples of the device having different elastic slider biasing members.

FIG. 6 illustrates an example of the device at the point where the camera contacts the wiper coupled to the slider and before contact with the ramp.

FIG. 7 illustrates an example of the device in a partially extended state.

FIG. 8 illustrates an example of the device in a camera pass through state.

FIG. 9 illustrates an example of the device illustrated in a wire diagram view.

FIG. 10 illustrates an example of the device in a camera extended state, where the device is illustrated in a wire diagram.

FIG. 11 illustrates an example cross-section of the device including the slider.

FIG. 12 illustrates an example of the device in which a wire spring is used to close the jaw toward the sheath.

FIG. 13 illustrates an example of the device being integrally formed with a trocar.

### DETAILED DESCRIPTION

It will be appreciated that for simplicity and clarity of illustration, where appropriate, reference numerals have been repeated among the different figures to indicate corresponding or analogous elements. In addition, while specific details are set forth in order to provide an understanding of the examples described herein, it will be appreciated by those skilled in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Several definitions that apply throughout this disclosure will now be presented. The term “coupled” is defined as connected, whether directly or indirectly through intervening components, and is not necessarily limited to physical connections. The term “communicatively coupled” is defined as connected, either directly or indirectly through intervening components, and the connections are not necessarily limited to physical connections, but are connections that accommodate the transfer of data between the so-described components. The connection can be such that the objects are permanently connected or releasably connected. The term “outside” refers to a region that is beyond the outermost confines of a physical object. The terms “comprising,” “including” and “having” are used interchangeably in this disclosure. The terms “comprising,” “including” and “having” mean to include, but not necessarily be limited to the things so described. Where components are described as being “operable to” perform certain operations, such configuration can be accomplished, for example, by designing hardware to perform the operation.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

In the field of laparoscopic or other minimally invasive surgery, the surgeon relies on a clear view of the surgical site within the abdominal cavity of the patient. The view is provided by a specialized cylindrical endoscopic or laparoscopic camera that passes through a tubular trocar. The present disclosure uses the phrase camera herein and the camera can be an endoscopic, laparoscopic or other suitable camera. The trocar is placed in the abdominal wall of the patient to provide a pathway from outside of the patent into the abdominal cavity. The trocar also provides a pathway where the cylindrical camera can be inserted rotated and manipulated as required to view the surgical site.

The lens of the camera can become contaminated with surgical debris, blood, or other liquids and/or solids. When this happens the lens must be cleaned. In order to clean the camera, the endoscope or laparoscope must be removed from the trocar and cleaned by a surgical assistant. Then, the surgical assistant must reinsert the endoscope or laparoscope before surgery can continue.

The present disclosure provides an improved version of a lens cleaning apparatus. The present disclosure has a key feature in that a wiper edge crosses the center of the camera lens. By crossing the centerline, a single wiper edge can sweep the entire view path of the camera and does not leave a residual band.

The device includes an attachment tube that is operable to be press fit onto a distal end of a trocar. The attachment tube can be operably configured for a desired trocar. The device can also include a flexible jaw member connecting the attachment tube to a jaw. The jaw can be operable to swing laterally to provide a path for the camera to pass therethrough. The jaw can be further equipped with a ramp riding on the passing camera. The device can further include a slider that moves axially along the jaw interior.

The device has a single jaw with a single slider. The single slider has a center crossing wiper. The device has an increased jaw angular travel to accommodate the increased lateral travel of the slider. The device has a sheath that in one example can extend from the attachment tube or be integrally formed with the attachment tube. The device can have an elastic member to draw tight to the jaw into the sheath.

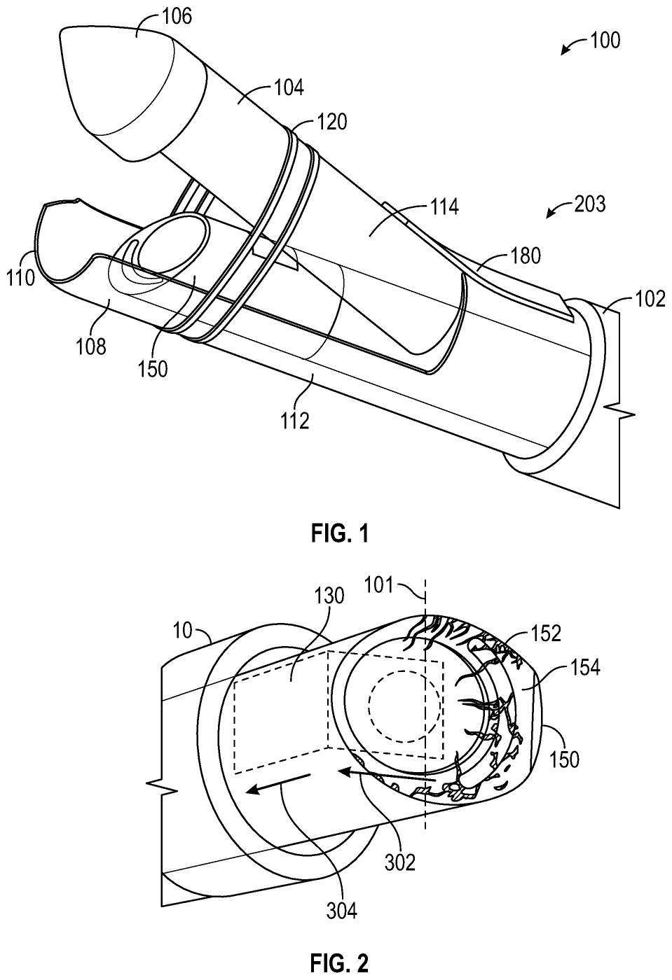

FIG. 1 illustrates an example device 100 with a camera 150 extended past the wiper blade (not shown) and the elastic member 120 in the extended state 203. The device 100 can be adapted to be fitted to an open end of a trocar tube. The device 100 can include a substantially cylindrical attachment tube 102 operable to be coupled to the open end of the trocar tube. The device 100 can also include a jaw 104 extending away from the attachment tube 102 and having a substantially conical distal end 106 located away from the attachment tube 102, wherein the jaw 104 has sidewalls 114 corresponding to a portion of the circumference of the tube 102. The device 100 can include a sheath 108 extending away from the attachment tube 102 and having a distal end 110 that extends toward the substantially conical distal end 106 of the jaw 104, wherein the sheath 108 has sidewalls 112 corresponding to another portion of the circumference of the tube 102 and the sidewalls 112 of the sheath 108 and the sidewalls 114 of the jaw 104 being biased towards each other through at least one elastic member 120.

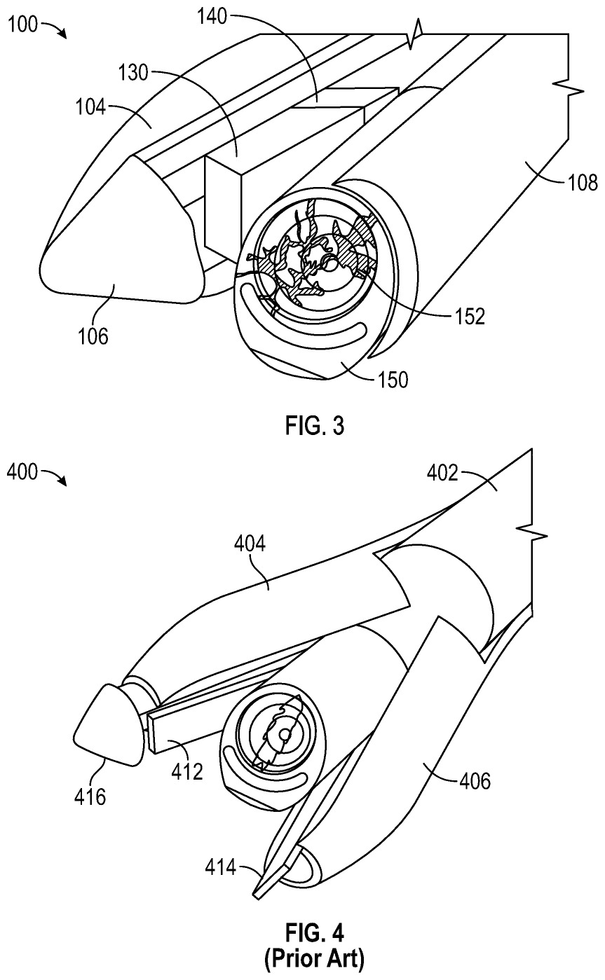

FIG. 2 illustrates a camera extending through a trocar 10 without the device attached thereto. As illustrated, the camera 150 can be positioned such that the slider 130 (shown in phantom as it is only for illustration) and associated wiper (not shown) covers the entirety or a substantial majority of the lens face 154 of the camera 150. As shown, the camera 150 lens face 154 can have fluids or substances 152 that are collected on the camera 150 lens face 154. A transverse centerline 101 shows the point on the camera 150 beyond which the wiper is positioned. The distance beyond the transvers centerline 101 covered by the wiper allows it to clean the camera 150 lens face 154 without leaving fluids or substances 152. In general, the fluids and/or substances 152 can be bodily fluids, tissue, muscle, fat and the like that are found during surgical operations. These fluids and/or substances 152 can be difficult to clean. Additionally, conventionally the cleaning of the fluids and/or substances 152 requires the camera 150 to be removed from the body cavity of the patient thereby causing a temperature change and further making it difficult to clean the camera 150 lens face 154. As shown, the slider 130 can be designed to move in an upward direction 302 past the transverse centerline 101. Additionally, the slider 130 can be configured to be pushed in a direction 304 relative to camera 150. As will be explained herein, the device 100 can be coupled to the trocar 10 or the device 100 can be integrally formed with the trocar 10. If the device 100 is coupled to the trocar 10, it allows the device to be sold separately for a variety of different trocars 10.

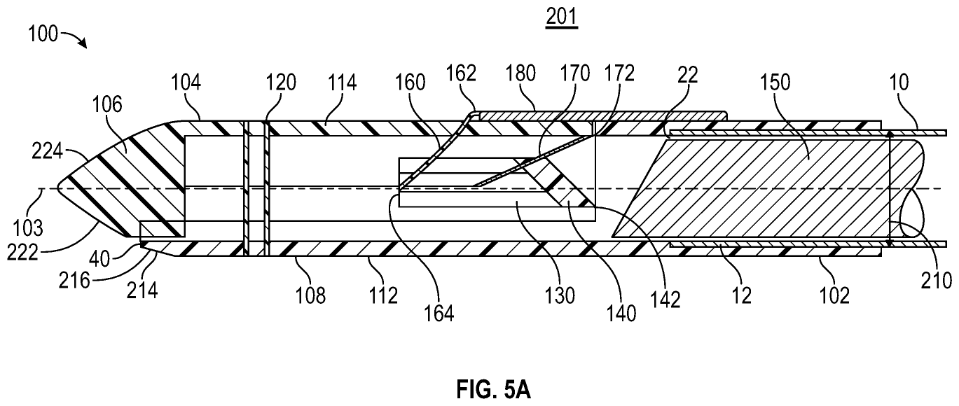

FIG. 3 illustrates a camera 150 that has been contaminated with fluids and/or substances 152 and being retracted for cleaning. The device 100 as shown is operable to include a sheath 108 having a distal end 110 and a jaw 104 having a substantially conical distal end 106. The device includes a slider 130 on which a wiper 140 is coupled. The wiper 140 can be of a different material from the slider. This allows the wiper 140 to be a material that is designed to remove the fluids and/or substances 152 from the camera 150. Details regarding the operation of the slider 130, wiper 140, and jaw 104 are described below. The present disclosure contemplates the implementation of one or more of the features described herein in regards to one example to be implemented with respect to other ones of the example. For example, the elastic member can be implemented on all of the devices 100. In other examples, the elastic member can be replaced with a wire spring while retaining other features as described herein.

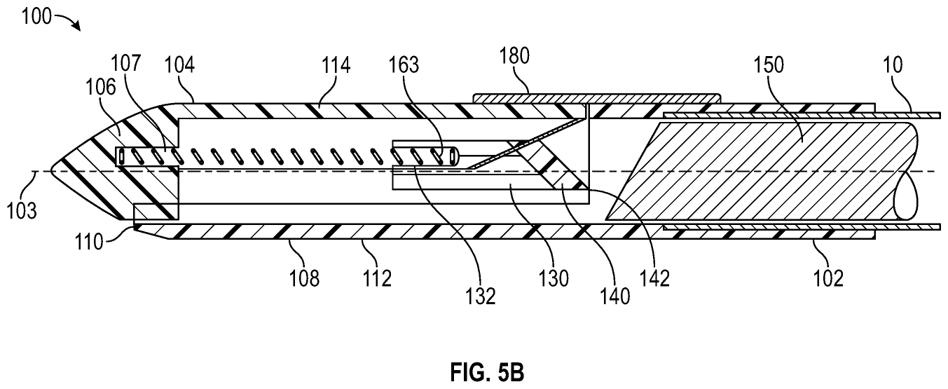

FIG. 4 illustrates the contamination that is left in a twin blade design of the prior art. The twin blade design included a first blade 412 and a second blade 414 coupled respectfully to a first opening member 404 and a second opening member 406. The first blade 412 had a tip 416 that was coupled thereto. In the illustrated example, the first opening member 404 and the second opening member 406 are coupled to a tube 402 of a trocar.

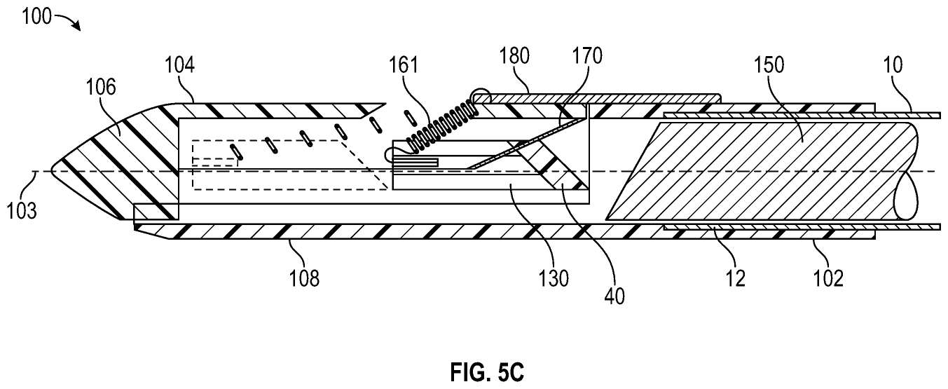

FIGS. 5A-5C illustrate examples of the device having different elastic slider biasing members. The device 100 can include a slider 130 coupled to the jaw 104 and operable to translate with respect to the jaw 104. The device 100 can be adapted to be fitted to an open end 22 of a trocar 12 tube. The device 100 can include a substantially cylindrical attachment tube 102 operable to be coupled to the open end 22 of the trocar tube 12. The device 100 can also include a jaw 104 extending away from the attachment tube 102 and having a substantially conical distal end 106 located away from the attachment tube 102, wherein the jaw 104 has sidewalls 114 corresponding to a portion of the circumference of the tube 102. The device 100 can include a sheath 108 extending away from the attachment tube 102 and having a distal end 110 that extends toward the substantially conical distal end 106 of the jaw 104, wherein the sheath 108 has sidewalls 112 corresponding to another portion of the circumference of the tube 102 and the sidewalls 112 of the sheath 108 and the sidewalls 114 of the jaw 104 being biased towards each other through at least one elastic member 120.

A wiper blade 140 can be coupled to the slider 130. The wiper blade 140 can have a lower edge 142 that extends away from the jaw 104 and extends beyond a centerline 103 with respect to the attachment tube 102. The wiper blade 140 can be operable to be engaged by a camera 150 traversing through the attachment tube 102, whereby the wiper blade 140 crosses the centerline 103 and allows the camera 150 to pass beyond the wiper 140, which is operable to remove fluids and/or substances (not shown) from the camera 150. The wiper blade 140 can be biased towards the camera 150 by an elastic slider biasing member 160. An elastic member 120 can bias the jaw 104 towards the sheath 108. Additionally, a flexible jaw member 180 can allow the jaw 104 to pivot relative attachment tube 102. In at least one example, the flexible jaw member 180 can bias the jaw 104 towards the sheath 108.

The at least one elastic member 120 as illustrated can be a rubber or metallic member. In some examples, the metallic member is desirable because of the ability to be autoclaved whereby the device 100 can be reused. In other examples, the rubber and/or polymer at least one elastic member 120 can be used to reduce costs so that the device 100 can be disposed of in accordance with normal standards. As illustrated, the at least one elastic member 120 includes two elastic members. In other examples, a single elastic member 120 can be implemented.

The device 100 can include a sheath 108. The sheath 108 can extend to partially overlap with the conical distal end 106 of the jaw 104. The sheath 108 can have a tapered portion 214 at the distal end 110 that has an outer slope 216 substantially identical to the slope 222 of the conical distal end 106 of the jaw 104. The substantially conical distal end 106 of the jaw can have a chisel like shape 224.

The device 100 can include an elastic slider biasing member 160 operable to bias the wiper 140 towards the opening in the attachment tube 102. The elastic slider biasing member 160 is illustrated in FIG. 5A as a polymer that has elastic properties. The elastic slider biasing member 160 can be coupled to the flexible jaw member 180 at a first end 162 and the slider 130 at a second end 164.

In other examples, the elastic slider biasing member 160 can take the form of a compression spring 163, FIG. 5B. In FIG. 5B, the elastic slider biasing member 160 is a compression spring 163 and is operable to be received within a cavity 107 formed in the substantially conical distal end 106 of the jaw 104 and a cavity 132 formed in the slider 130.

In yet other examples, the elastic slider biasing member 160 can take the form of a metal tension spring 161, FIG. 5C. While these examples are shown as mutually exclusive in the figures, the present disclosure contemplates that elastic slider biasing member 160 can include one or more of the different examples as described herein.

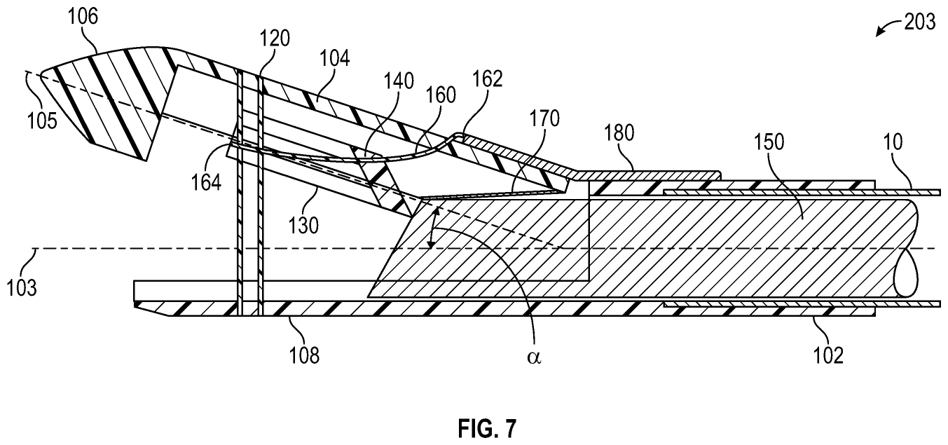

As shown in FIGS. 5A-5C, the device 100 can have a ramp 170 that is coupled to the jaw 104, wherein the ramp 170 is operable to be contacted by the camera 150 and cause the jaw 104 to be opened, as illustrated in FIG. 7. The ramp 170 can be integrally formed with the jaw 104. The end 172 of the ramp 170 closest to the attachment tube 102 is located closer to the attachment tube 102 than the wiper blade 140 in a retracted state 201, the wiper blade 140 can be positioned in the retracted state 201 to contact the camera 150 prior to the camera 150 contacting the ramp 170. In one example, the ramp 170 can be coupled between the slider 130 and the jaw 104.

The device 100 can have a flexible jaw member 180 coupled to the jaw 104. The flexible jaw member 180 can be operable to allow the jaw 104 to move relative to the attachment tube 102. The substantially cylindrical attachment tube 102 can have an inner diameter 210 correspondingly sized to provide a press fit connection with the open end 22 of the trocar tube 12.

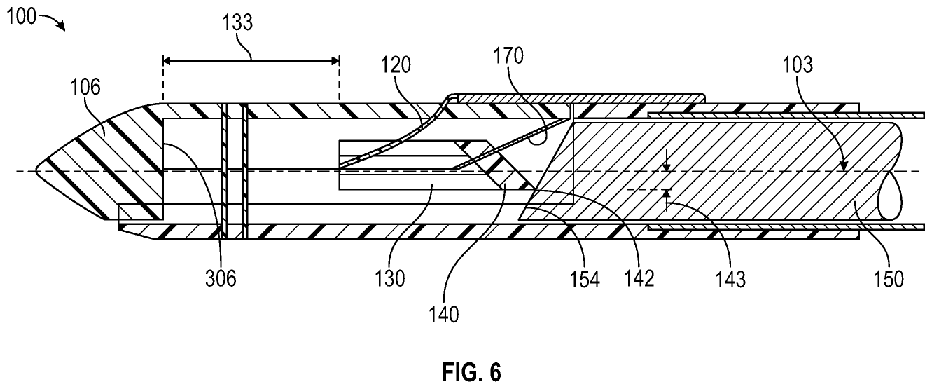

FIG. 6 illustrates an example of the device 100 at the point where the camera 150 contacts the wiper 140 coupled to the slider 130 and before the camera 150 contacts the ramp 170. As illustrated, the elastic slider member 120 pulls the slider 130 towards the camera 150 so that the wiper 140 contacts the camera lens face 154. As shown the lower edge 142 of the wiper contact the camera lens face 154. The position in which the wiper 140 contacts the camera lens face 154 is such that the opposite end of the slider 130 is a distance 133 away from an inner surface 306 of the conical distal end 106 of the jaw 104. The lower edge 142 of the wiper is located a distance 143 below the centerline 103 of the attachment tube 102 relative to the jaw 104.

FIG. 7 illustrates an example of the device 100 in a partially extended state 203. As illustrated, the device 100 has a flexible jaw member 180 coupled to the jaw 104. The flexible jaw member 180 is operable to allow the jaw 104 to move relative to the attachment tube 102. As shown, an angle α can be formed between the centerline 103 and a plane of the jaw 105. The elastic slider biasing member 160 is coupled to the flexible jaw member 180 at a first end 162 and the slider 130 at a second end 164. The slider 130 is operable to slide relative to the jaw 104 in substantially the same plane 105 as the jaw 104. The jaw 104 can continue opening relative to the attachment tube 102 until the camera passes beyond the slider 130 and wiper 140.

As the jaw 104 opens, the elastic member 120 pulls the jaw 104 towards the sheath 108. As illustrated, the plane 105 of the jaw 104 extending through the tip of the conical distal end 106 is such that it forms an angle α with respect to the centerline 103 of the attachment tube 102.

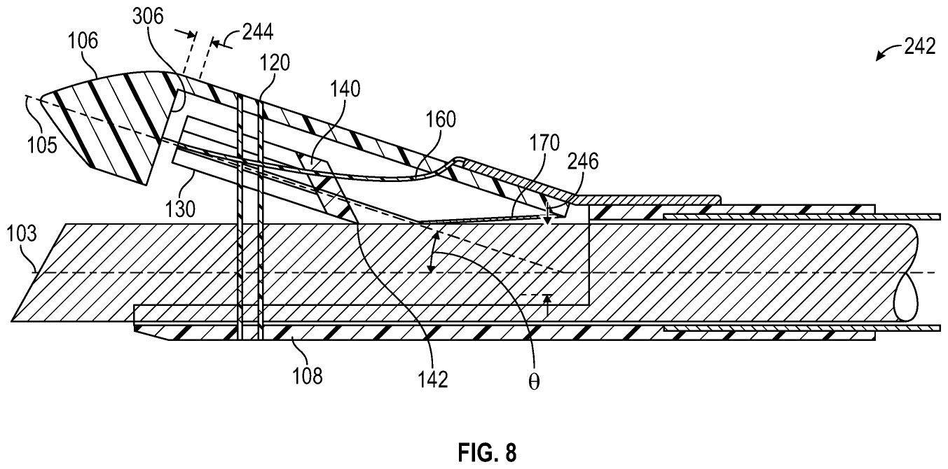

FIG. 8 illustrates an example of the device 100 in a camera pass through state 242. As illustrated, the slider extends such that a distance 244 is formed between the slider 130 and the conical distal end 106 of the jaw 104. As shown, the elastic members 120 are in a camera pass through state 242 and are forcing the jaw 104 towards the sheath 108. Additionally, the total distance of travel of the lower edge 142 of the wiper blade 142 can be described as a camera traverse distance 246. As shown the ramp 170 is in contact with an upper edge of the camera. Furthermore, the elastic slider biasing member 160 is extended even further than as illustrated in FIG. 7. An angle θ is formed between the plane 105 of the jaw 104 passing through the tip of the conical distal end 106 and the centerline 103 of the attachment tube 102.

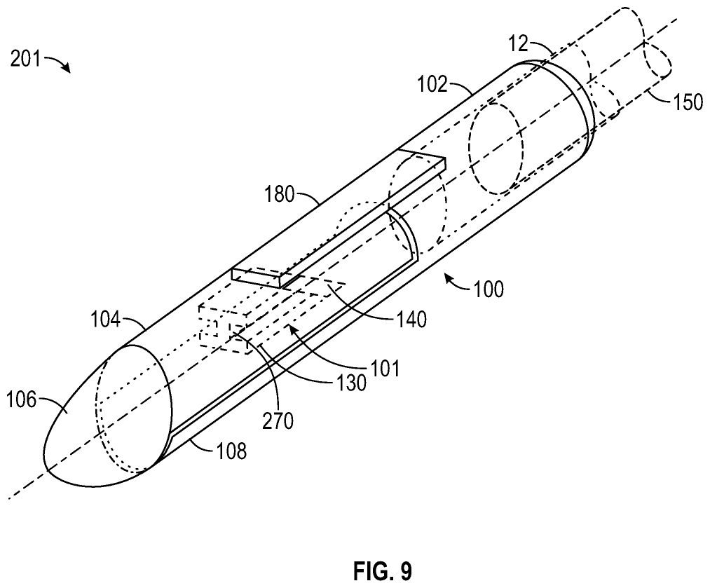

FIG. 9 illustrates an example of the device 100 illustrated in a wire diagram view in a retracted state 201. As seen, the device includes a flexible jaw member 180 located between the jaw 104 and the attachment tube 102. The jaw 104 is located above the sheath 108. The attachment tube is coupled to the trocar tube 12. The slider 130 is shown in dashed lines and illustrates one example of at least one channel 270 being formed in the slider 130. The at least one channel 270 is operable to allow the slider 130 to move relative to the jaw 104. The jaw 104 can have a corresponding protrusion portion (not shown) that extends into the at least one channel 270.

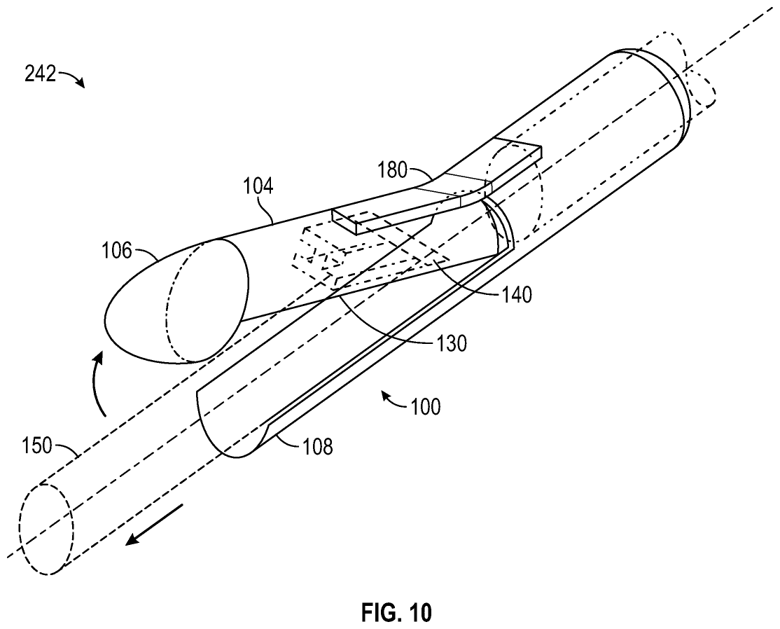

FIG. 10 illustrates an example of the device 100 in a camera extended state 242, where the device 100 is illustrated in a wire diagram. As illustrated, the camera 150 is extended beyond the device 100. The slider 130 and wiper 140 are positioned such that the wiper 140 is in contact with the outer surface of the camera 150 that is closest to the jaw 104. As illustrated the jaw 104 and the conical distal end 106 extend away from the sheath 108 to allow the camera 150 to pass therethrough. Additionally, the flexible jaw member 180 is bent to allow the jaw 104 to pivot relative to the attachment tube.

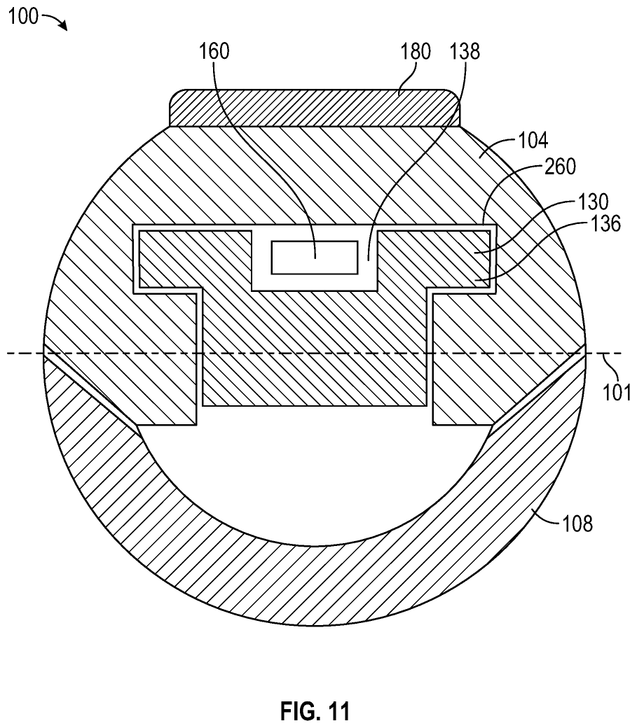

FIG. 11 illustrates an example cross-section of the device 100 including the slider 130. The example in FIG. 11 implements a different channel and protrusion arrangement as compared to FIGS. 9 and 10. The implementation of the different arrangements can be interchanged with the remainder of the description. As illustrated, the slider 130 includes protrusions 136 that are received in corresponding slots 260 formed in the jaw 104. Additionally, the slider 130 has a channel 138 formed therein to receive the elastic slider biasing member 160. As illustrated the flexible jaw member 180 is located on the top and the jaw 104 below it followed by the sheath. Additionally, the slider 130 extends below the transvers centerline 101. In extending below the transverse centerline, the slider allows the wiper to correspondingly extend the same distance below the transvers centerline.

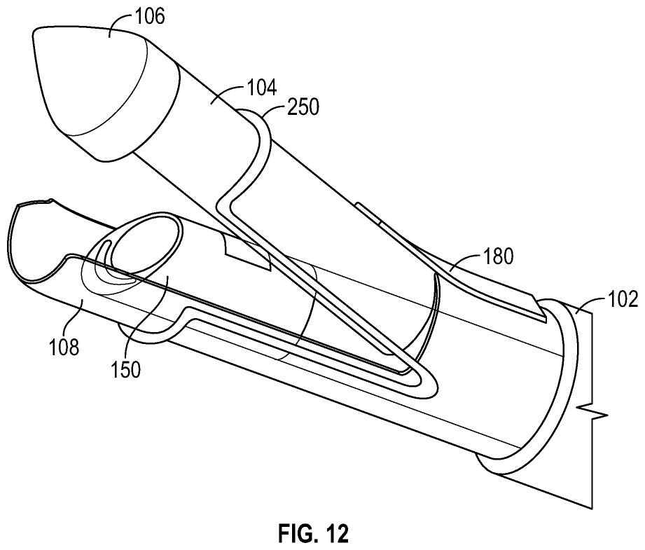

FIG. 12 illustrates an example of the device in which a wire spring 250 is used to close the jaw 104 toward the sheath 108. The wire spring 250 is used in place of the elastic members 120 shown in the previous figures. As illustrated, the conical distal end 106 extends away from the sheath 108 to allow the camera to pass therethrough. Other features of the device 100 can be implemented in this example as well.

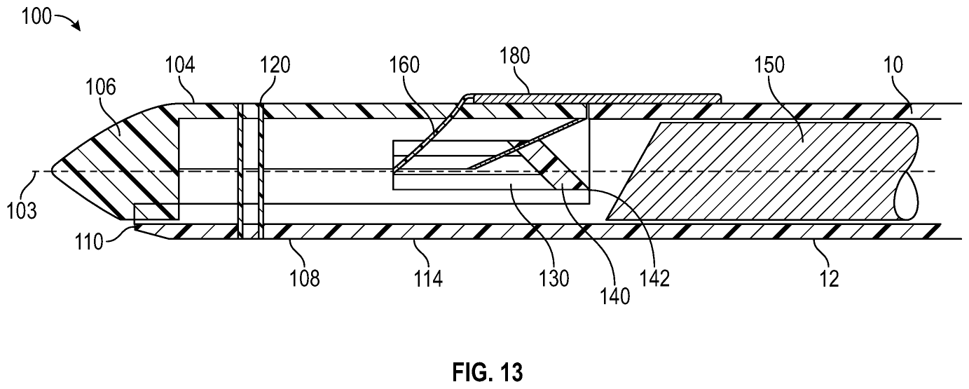

FIG. 13 illustrates an example of the device 100 being integrally formed with a trocar 10. While the example shown in FIG. 13 most closely resembles that of FIG. 5A, the implementation where the device 100 is integrally formed with the trocar 10 can be such that the other features as describe above can be implemented in place of the ones illustrated. As shown the trocar 10 includes a tube 12. The trocar 10 can also include a jaw 104 extending away from the tube 12 and having a substantially conical distal end 106 located away from the tube 12. The jaw 14 has sidewalls 114 corresponding to a portion of the circumference of the tube 12.

The trocar 10 can include a sheath 108 extending away from the tube 12 and having a distal end 110 that extends toward the substantially conical distal end 106 of the jaw 104. The sheath 108 has sidewalls 112 corresponding to another portion of the circumference of the tube 12 and the sidewalls 112 of the sheath and the sidewalls 114 of the jaw 104 being biased towards each other through at least one elastic member 120.

The trocar 10 can include a slider 130 coupled to the jaw 104 and operable to translate with respect to the jaw 104. Additionally, the trocar 10 can include a wiper blade 140 coupled to the slider 130. The wiper blade 140 can have a lower edge 142 that extends away from the jaw 104 and extends beyond a centerline 103 with respect to the tube 12. The wiper blade 140 is operable to be engaged by a camera 150 traversing through the tube 12. The wiper blade 140 crosses the centerline 103 and allows the camera 150 to pass beyond the wiper 140, which is operable to remove fluids and/or substances from the camera 150. As illustrated the device 100 includes the flexible jaw member 180. Furthermore, the wiper 140 can be biased towards the camera by the elastic slider biasing member 160. The other examples of the biasing members described herein can be used in place of the elastic slider biasing member 160.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

Illustrative examples of the disclosure include:
Aspect 1: A device alternatively being adapted to be coupled with a trocar comprising: an attachment tube operable to be coupled to the open end of the trocar tube; a jaw extending away from the attachment tube, wherein the jaw has sidewalls corresponding to a portion of the circumference of the tube; a sheath extending away from the attachment tube, wherein the sheath has sidewalls corresponding to another portion of the circumference of the tube and the sidewalls of the sheath and the sidewalls of the jaw being biased towards each other through at least one elastic member; a slider coupled to the jaw and operable to translate with respect to the jaw; a wiper blade coupled to the slider, wherein the wiper blade has a lower edge that extends away from the jaw and extends beyond a centerline with respect to the attachment tube; wherein the wiper blade is operable to be engaged by a camera traversing through the attachment tube, whereby the wiper blade crosses the centerline and allows the camera to pass beyond the wiper, which is operable to remove fluids and/or substances from the camera.
Aspect 2: A trocar device comprising: a tube operable; a jaw extending away from the tube, wherein the jaw has sidewalls corresponding to a portion of the circumference of the tube; a sheath extending away from the tube, wherein the sheath has sidewalls corresponding to another portion of the circumference of the tube and the sidewalls of the sheath and the sidewalls of the jaw being biased towards each other through at least one elastic member; a slider coupled to the jaw and operable to translate with respect to the jaw; a wiper blade coupled to the slider, wherein the wiper blade has a lower edge that extends away from the jaw and extends beyond a centerline with respect to the tube; wherein the wiper blade is operable to be engaged by a camera traversing through the tube, whereby the wiper blade crosses the centerline and allows the camera to pass beyond the wiper, which is operable to remove fluids and/or substances from the camera.
Aspect 3: The device of any of Aspects 1-2, wherein the tube is substantially cylinderal.
Aspect 4: The device of any of Aspects 1-3, wherein the jaw has a substantially conical distal end located away from the tube.
Aspect 5: The device of any of Aspects 1-4, wherein the sheath having a distal end that extends toward the substantially conical distal end of the jaw.
Aspect 6: The device of any of Aspects 1-5, further comprising an elastic slider biasing member operable to bias the wiper towards the opening in the tube.
Aspect 7: The device of Aspect 6, further comprising a ramp that is coupled to the jaw, wherein the ramp is operable to be contacted by the camera and cause the jaw to be opened.
Aspect 8: The device of Aspect 7, wherein the ramp is integrally formed with the jaw.
Aspect 9: The device of Aspect 7, wherein the end of the ramp closest to the tube is located closer to the tube than the wiper blade in a retracted state, the wiper blade being positioned in the retracted state to contact the camera prior to the camera contacting the ramp.
Aspect 10: The device of Aspect 7, wherein the ramp is coupled between the slider and the jaw.
Aspect 11: The device of Aspect 6, further comprising a flexible jaw member coupled to the jaw, wherein the flexible jaw member is operable to allow the jaw to move relative to the tube.
Aspect 12: The device of Aspect 11, wherein the elastic slider biasing member is coupled to the flexible jaw member at a first end and the slider at a second end and the slider is operable to slide relative to the jaw in substantially the same plane as the jaw.
Aspect 13: The device of Aspect 12, wherein the elastic slider biasing member is a metal tension spring.
Aspect 14: The device of Aspect 12, wherein the elastic slider biasing member is a compression spring and is operable to be received within a cavity formed in the substantially conical distal end of the jaw and a cavity formed in the slider.
Aspect 15: The device of any Aspects 1-14, wherein the substantially cylindrical tube has an inner diameter correspondingly sized to provide a press fit connection with the open end of the trocar tube.
Aspect 16: The device of any Aspects 1-15, wherein the at least one elastic member comprises two elastic members.
Aspect 17: The device of any Aspects 1-16, wherein the at least one elastic member is made of rubber.
Aspect 18: The device of any Aspects 1-17, wherein the at least one elastic member is metallic.
Aspect 19: The device of any Aspects 1-18, wherein the sheath extends to partially overlap with the conical distal end of the jaw.
Aspect 20: The device of Aspect 19, wherein the sheath has a tapered portion at the distal end that has an outer slope substantially identical to the slope of the conical distal end of the jaw.
Aspect 21: The device of any Aspects 1-20, wherein the substantially conical distal end has a chisel like shape.

1. A device adapted to be fitted to an open end of a trocar tube, the device comprising:
a substantially cylindrical attachment tube operable to be coupled to the open end of the trocar tube;
a jaw extending away from the attachment tube and having a substantially conical distal end located away from the attachment tube, wherein the jaw has sidewalls corresponding to a portion of a circumference of the tube;
a sheath extending away from the attachment tube and having a distal end that extends toward the substantially conical distal end of the jaw, wherein the sheath has sidewalls corresponding to another portion of the circumference of the tube and the sidewalls of the sheath and the sidewalls of the jaw being biased towards each other through at least one elastic member;
a slider coupled to the jaw and operable to translate with respect to the jaw;
a wiper blade coupled to the slider, wherein the wiper blade has a lower edge that extends away from the jaw and extends beyond a centerline with respect to the attachment tube;
wherein the wiper blade is operable to be engaged by a camera traversing through the attachment tube, whereby the wiper blade crosses the centerline and allows the camera to pass beyond the wiper, which is operable to remove fluids and/or substances from the camera.

2. The device as recited in claim 1, further comprising an elastic slider biasing member operable to bias the wiper towards the opening in the attachment tube.

3. The device as recited in claim 2, further comprising a ramp that is coupled to the jaw, wherein the ramp is operable to be contacted by the camera and cause the jaw to be opened.

4. The device as recited in claim 3, wherein the ramp is integrally formed with the jaw.

5. The device as recited in claim 3, wherein the end of the ramp closest to the attachment tube is located closer to the attachment tube than the wiper blade in a retracted state, the wiper blade being positioned in the retracted state to contact the camera prior to the camera contacting the ramp.

6. The device as recited in claim 3, wherein the ramp is coupled between the slider and the jaw.

7. The device of claim 2, further comprising a flexible jaw member coupled to the jaw, wherein the flexible jaw member is operable to allow the jaw to move relative to the attachment tube.

8. The device of claim 7, wherein the elastic slider biasing member is coupled to the flexible jaw member at a first end and the slider at a second end and the slider is operable to slide relative to the jaw in substantially the same plane as the jaw.

9. The device of claim 8, wherein the elastic slider biasing member is a metal tension spring.

10. The device of claim 8, wherein the elastic slider biasing member is a compression spring and is operable to be received within a cavity formed in the substantially conical distal end of the jaw and a cavity formed in the slider.

11. The device of claim 1, wherein the substantially cylindrical attachment tube has an inner diameter correspondingly sized to provide a press fit connection with the open end of the trocar tube.

12. The device of claim 1, wherein the at least one elastic member comprises two elastic members.

13. The device of claim 1, wherein the at least one elastic member is made of rubber.

14. The device of claim 1, wherein the at least one elastic member is metallic.

15. The device of claim 1, wherein the sheath extends to partially overlap with the conical distal end of the jaw.

16. The device of claim 15, wherein the sheath has a tapered portion at the distal end that has an outer slope substantially identical to the slope of the conical distal end of the jaw.

17. The device as recited in claim 1, wherein the substantially conical distal end has a chisel like shape.

18. A trocar comprising:
a tube;
a jaw extending away from the tube and having a substantially conical distal end located away from the tube, wherein the jaw has sidewalls corresponding to a portion of a circumference of the tube;
a sheath extending away from the tube and having a distal end that extends toward the substantially conical distal end of the jaw, wherein the sheath has sidewalls corresponding to another portion of the circumference of the tube and the sidewalls of the sheath and the sidewalls of the jaw being biased towards each other through at least one elastic member;
a slider coupled to the jaw and operable to translate with respect to the jaw;
a wiper blade coupled to the slider, wherein the wiper blade has a lower edge that extends away from the jaw and extends beyond a centerline with respect to the tube;
wherein the wiper blade is operable to be engaged by a camera traversing through the tube, whereby the wiper blade crosses the centerline and allows the camera to pass beyond the wiper, which is operable to remove fluids and/or substances from the camera.
